(19) United States
(12) Patent Application Publication
(10) Pub. No.: US 2026/0167592 A1
(43) Pub. Date: Jun. 18, 2026

(54) EFFICIENT OXIDATIVE CHLORINATION AND BROMINATION METHOD OF OLEFINS WITH SELECTFLUOR AND TBAX (Cl, Br)

(71) Applicant: ATATÜRK ÜNIVERSITESI FIKRI MÜLKIYET HAKLARI KOORDINATÖRLÜGÜ DÖNER SERMAYE ISLETMESI, ERZURUM (TR)

(72) Inventors: BILAL NISANCI, Erzurum (TR); ZIYA DAGALAN, Erzurum (TR); ARIF DASTAN, Erzurum (TR); RAMAZAN KOÇAK, Erzurum (TR)

(73) Assignee: TATÜRK ÜNIVERSITESI FIKRI MÜLKIYET HAKLARI KOORDINATÖRLÜGÜ DÖNER SERMAYE ISLETMESI, ERZURUM (TR)

(21) Appl. No.: 18/713,206

(22) PCT Filed: Dec. 12, 2023
(86) PCT No.: PCT/TR2023/051523

§ 371 Date: May 24, 2024

(30) Foreign Application Priority Data
Dec. 13, 2022 (TR) 2022/019229

(51) Int. Cl. C07C 17/02

(52) U.S. Cl. CPC C07C 17/02

(57) ABSTRACT

The present invention relates to a method for chlorination and bromination reactions of olefins using environmentally friendly TBAX (Br, Cl) and Selectfluor salts as an alternative to metal and toxic halogen sources for chlorination and bromination reactions of olefins, eliminating the problems caused by classical halogenation methods, metal-free, non-toxic and simple operating conditions.

### FIELD OF THE INVENTION

This invention eliminates the need for the use of metal halides and toxic molecular halogenation reagents (Cl₂, Br₂) for chlorination and bromination reactions of olefins, and provides an alternative methodology by using environmentally friendly TBAX (Br, Cl) and Selectfluor salts, which eliminates the problems of classical halogenation methods, is metal-free, non-toxic and has simple operating conditions.

### STATE OF THE ART

Although halogenated compounds are themselves directly valuable, they are also key starting molecules in many derivatization reactions such as substitution, elimination, coupling and metallation (Mg, Li, Cu, etc.). Almost all multistep synthesis in organic chemistry involve halogenated starting compounds or intermediates. Therefore, the halogenation of organic molecules and the synthesis of halogenated derivatives has been a topic of research for many years, but it is still very relevant and important. Among halides, chlorides and bromides are more prominent than iodides and fluorides due to their great commercial importance.

Molecular halogenation reagents (Cl₂, Br₂) and metal halides (MXn) are generally used for the halogenation of olefins. Molecular halogenation reagents (Cl₂, Br₂) are extremely toxic and have harsh working conditions. On the other hand, metal halides are not preferred halogen sources for medicinal chemistry because they contain metals. Therefore, there is a need for new metal-free, non-toxic and simple operating conditions for chlorination and bromination reactions.

In the state of the art, molecular halogen sources and metal halides are generally used for the halogenation of olefins. The use of molecular halogen sources or metal halides has many negative aspects and disadvantages such as the need for extremely toxic molecular chlorine gas and bromine reagent, the metal content, the lack of stabilization in high yield and selectivity, the inability to derivatize natural chiral compounds, and the inability to use safe solvents depending on the conditions.

The advantages of the chlorination and bromination method with TBA⁺X⁻ and selectfluor without using molecular halogenation reagents (Cl₂, Br₂) and metal halides (MXn) are not available in the methods known in the literature.

### BRIEF DESCRIPTION OF THE INVENTION

The present invention relates to an efficient method for the synthesis of chlorinated and brominated halogen compounds which meets the above-mentioned requirements, eliminates all disadvantages, does not require the extremely toxic molecular chlorine gas and bromine reagent, does not contain any metal, enables the synthesis of halogenated products in high yield and selectivity, allows the derivatization of natural chiral compounds and allows the use of safe solvents in mild conditions.

The primary purpose of the invention is to use environmentally friendly TBAX (Br, Cl) and Selectfluor salts for the halogenation of olefins as an alternative to metal and toxic halogen sources, instead of the generally used molecular halogenation reagents (Cl2, Br2) and metal halides (MXn).

Another aim of the invention is to eliminate the problems created by classical halogenation methods.

Another aim of the invention is to provide a method with simple operating conditions.

The structural and characteristic features of the present invention will be understood clearly by the following detailed description. Therefore the evaluation shall be made by taking this detailed description into consideration.

### DETAILED DESCRIPTION OF THE INVENTION

In this detailed description, the efficient oxidative chlorination and bromination of olefins in the presence of Selectfluor and TBAX (Cl, Br) is presented in a non-limiting manner for a better understanding of the subject.

The invention is a method for efficient oxidative chlorination and bromination of olefins in the presence of Selectfluor and TBAX (Cl, Br).

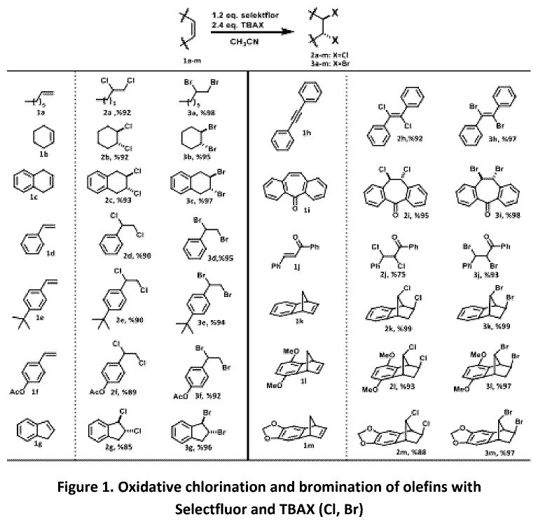

FIG. 1 shows the method of oxidative chlorination and bromination of olefins with Selectfluor and TBAX (Cl, Br).

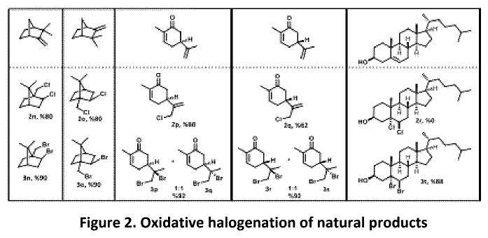

FIG. 2 shows the oxidative halogenation of natural products.

Olefin (1) (0.5 mmol), Selectfluor (209 mg, 0.6 mmol) and TBAX (1.2 mmol) were dissolved in 2 mL CH₃CN in a thermolysis tube, the reaction mixture was stirred magnetically, the solvent was removed in an evaporator and the crude product was purified by column chromatography on silica gel (n-hexane/EtOAc (9:1)).

For chlorination; Olefin (1) (0.5 mmol), Selectfluor (209 mg, 0.6 mmol) and TBACl (333 mmol, 1.2 mmol) were dissolved in 2 mL CH₃CN in a thermolysis tube, the reaction mixture was stirred magnetically at 100° C., the solvent was removed in an evaporator and the crude product was purified by column chromatography on silica gel (n-hexane/EtOAc (9:1)).

For bromination; Olefin (1) (0.5 mmol), Selectfluor (218 mg, 0.6 mmol) and TBABr (386 mg, 1. 2 mmol)) in 2 mL CH₃CN in a thermolysis tube, the reaction mixture was stirred at room temperature for 5 min (for 1i and 1j: 24 h), the solvent was removed in an evaporator and the crude product was purified by column chromatography on silica gel (n-hexane/EtOAc (9:1)).

### 1,2-Dichlorooctane (2a)¹

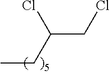

Colorless oil (91 mg, 92%). 1H-NMR (400 M Hz, CDCl₃) δ 4.11-3.97 (m, 1H), 3.76 (dd, J=11.3, 5.2 Hz, 1H), 3.65 (dd, J=11.3, 7.4 Hz, 1H), 2.07-1.92 (m, 1H), 1.76-1.64 (m, 1H), 1.37-1.16 (m, 8H), 0.89 (t, J=6.7 Hz, 3H). ¹³C NMR (101 MHz, CDCl₃) δ 61.4, 48.4, 35.2, 31.7, 28.8, 25.9, 22.7, 14.2.

### trans-1,2-Dichlorocyclohexane (2b)¹

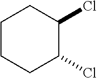

Colorless oil (70 mg, 92%). ¹H NMR (400 MHz, CDCl₃) δ 4.13-3.95 (m, 2H), 2.42-2.21 (m, 2H), 1.92-1.66 (m, 4H), 1.51-1.35 (m, 2H). ¹³C NMR (101 MHz, CDCl₃) δ 63.3, 33.5, 23.2.

### trans-2,3-Dichloro-1,2,3,4-tetrahydronaphthalene (2c)²

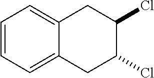 [SMILES: Cl[C@@H]1Cc2ccccc2C[C@H]1Cl]

Colorless oil (93 mg, 93%). ¹H NMR (400 MHz, CDCl₃) δ 7.23-7.16 (AA′ part of AA′BB′ system, 2H), 7.14-7.07 (BB′ part of AA′BB′ system, 2H), 4.50-4.44 (m, 2H), 3.67 (dd, A part of AB system, J=18.2, 3.2 Hz, 2H), 3.13 (bd, B part of AB system, J=18.2 Hz, 2H). ¹³C NMR (101 MHz, CDCl₃) δ 131.3, 129.0, 126.9, 58.0, 34.8.

### (1,2-Dikloroetil)benzen (2d)³

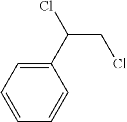

Colorless oil (78 mg, 92%). ¹H NMR (400 MHz, CDCl₃) δ 7.48-7.33 (m, 5H), 5.01 (dd, J=7.9, 6.6 Hz, 1H), 4.01 (dd, J=11.3, 6.6 Hz, 1H), 3.94 (dd, J=11.3, 7.9 Hz, 1H). ¹³C NMR (101 MHz, CDCl₃) δ 138.1, 129.3, 129.0, 127.5, 61.9, 48.5.

### 1-(tert-Butyl)-4-(1,2-dichloroethyl)benzene (2e)³

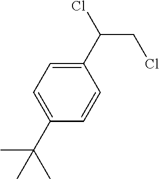

Colorless oil (104 mg, 90%). ¹H NMR (400 MHz, CDCl₃) δ 7.41 (d, J=8.4 Hz, 2H), 7.34 (d, J=8.4 Hz, 2H), 5.00 (t, J=7.2 Hz, 1H), 4.02-3.90 (m, 2H), 1.33 (s, 3H). ¹³C NMR (101 MHz, CDCl₃) δ 152.4, 135.1, 127.2, 125.9, 62.0, 48.6, 34.8, 31.4.

### 4-(1,2-Dichloroethyl)phenyl acetate 2 (f)⁴

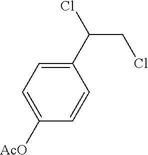 [SMILES: CC(=O)Oc1ccc(C(Cl)CCl)cc1]

Colorless oil (103 mg, 89%). ¹H NMR (400 MHz, CDCl₃) δ 7.42 (d, J=8.7 Hz, 2H), 7.12 (d, J=8.7 Hz, 2H), 5.11-4.90 (m, 1H), 3.97 (dd, J=11.4, 6.6 Hz, 1H), 3.89 (dd, J=11.4, 7.8 Hz, 1H), 2.29 (s, 3H). ¹³C NMR (101 MHz, CDCl₃) δ 169.2, 151.1, 135.5, 128.6, 122.0, 61.2, 48.4, 21.1.

### trans-1,2-Dichloro-2,3-dihydro-1H-indene (2g)³

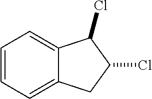 [SMILES: Cl[C@@H]1Cc2ccccc2[C@H]1Cl]

Colorless oil (79 mg, 85%). ¹H NMR (400 MHz, CDCl₃) δ 7.48-7.43 (m, 1H), 7.40-7.23 (m, 3H), 5.35 (d, J=2.8 Hz, 1H), 4.66 (dt, J=6.1, 3.2 Hz, 1H), 3.71 (dd, J=16.8, 6.1 Hz, 1H), 3.19 (dd, J=16.8, 3.2 Hz, 1H). ¹³C NMR (101 MHz, CDCl₃) δ 140.0, 129.8, 128.1, 125.6, 125.2, 67.8, 64.6, 40.9 2C signal coincident).

### (E)-1,2-Dichloro-1,2-diphenylethene (2h)⁵

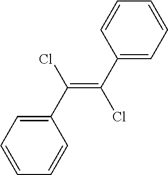 [SMILES: Cl/C(=C(/Cl)c1ccccc1)c1ccccc1]

White solid (114 mg, 92%). EN.: 92-94° C. ¹H NMR (400 MHz, CDCl₃) δ 7.66-7.59 (m, 4H), 7.50-7.36 (m, 6H). ¹³C NMR (101 MHz, CDCl₃) δ 137.7, 129.3, 129.2, 128.4, 127.9.

### trans-10,11-Dichloro-10,11-dihydro-5H-dibenzo[a,d][7]annulen-5-one (2i)

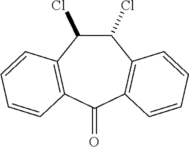 [SMILES: O=C1c2ccccc2[C@@H](Cl)[C@H](Cl)c2ccccc21]

Light yellow solid (131 mg, 95%). EN: 182-184° C. ¹H NMR (400 MHz, CDCh) δ 8.05 (dd, J=7.7, 1.1 Hz, 2H), 7.57 (dt, J=7.5, 1.4 Hz, 2H), 7.50 (dt, J=7.5, 1.2 Hz, 2H), 7.43 (dd, J=7.5, 1.1 Hz, 2H), 5.58 (s, 2H). ¹³C NMR (101 MHz, CDCl₃) δ 192.8, 137.9, 135.4, 132.9, 131.5, 131.2, 129.8, 62.4. HRMS (Q-TOF): m/z [M+H]⁺ Calculated C₁₅H₁₁Cl₂O: 277.0182, found: 277.0182.

### 2,3-Dichloro-1,3-dyphenlypropane-1-one (2j)⁴

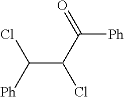 [SMILES: O=C(c1ccccc1)C(Cl)C(Cl)c1ccccc1]

White solid (104 mg, 75%). EN: 115-117° C. ¹H NMR (400 MHz, CDCl₃) δ 8.15-8.02 (m, 2H), 7.71-7.63 (m, 1H), 7.60-7.50 (m, 4H), 7.51-7.37 (m, 3H), 5.51 (d, J=10.6 Hz, 1H), 5.48 (d, J=10.6 Hz, 1H). ¹³C NMR (101 MHz, CDCl₃) δ 191.5, 137.2, 134.9, 134.4, 129.5, 129.2 (2C), 128.9, 128.5, 60.2, 57.2.

### (1R(S),2S(R),4R(S),9R(S))-2,9-dichloro-1,2,3,4-tetrahydro-1,4-methanonaphthalene (2k)⁶

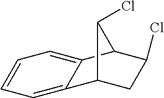 [SMILES: ClC1CC2c3ccccc3C1C2Cl]

White solid (102 mg, 99%). EN: 88-90° C. ¹H NMR (400 MHz, CDCl₃) δ 7.25-7.12 (m, 4H), 4.12-4.08 (m, 1H), 3.86 (ddd, J=7.9, 4.3, 1.1 Hz, 1H), 3.66-3.63 (m, 1H), 3.53-3.49 (m, 1H), 2.70 (dt, part A of system AB, J=13.2, 4.0 Hz, 1H), 2.19 (dd, part B of system AB, J=13.2, 7.9 Hz, 1H). ¹³C NMR (101 MHz, CDCl₃) δ 144.0, 142.3, 128.0, 127.4, 122.1, 122.0, 66.4, 56.9, 56.9, 50.6, 36.4.

### 2I. (1R(S),2S(R),4R(S),9R(S))-2,9-dichloro-5,8-dimethoxy-1,2,3,4-tetrahydro-1,4-methanonaphthalene (2I)

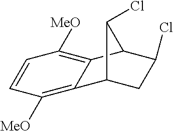 [SMILES: COc1ccc(OC)c2c1C1CC(Cl)C2C1Cl]

brown oil (127 mg, 93%). ¹H NMR (400 MHz, CDCl₃) δ 6.65 (d, J=9.0 Hz, 1H), 6.62 (d, J=9.0 Hz, 1H), 4.08-4.04 (m, 1H), 3.87-3.82 (m, 2H), 3.78 (s, 3H), 3.76 (s, 3H), 3.70-3.66 (m, 1H), 2.65 (dt, part A of system AB, J=13.2, 4.0 Hz, 1H), 2.18 (dd, part B of system AB, J=13.2, 7.9 Hz, 1H). ¹³C NMR (101 MHz, CDCl₃) δ 147.9, 147.7, 133.0, 131.0, 111.1, 110.2, 66.4, 56.6, 56.1, 55.9, 53.1, 47.1, 35.9. HRMS (Q-TOF): m/z [M]⁺ Calculated C₁₃H₁₄Cl₂O₂: 272.0365, found: 272,0356.

### (5R(S),6S(R),8R(S),10R(S))-6,10-dichloro-5,6,7,8-tetrahydro-5,8-methanonaphtho[2,3-d][1,3]dioxol (2m)

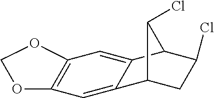 [SMILES: ClC1CC2c3cc4c(cc3C1C2Cl)OCO4]

White solid (113 mg, 88%). EN: 101-103° C. ¹H NMR (400 MHz, CDCl₃) δ 6.73 (s, 1H), 6.69 (s, 1H), 5.92 (d, J=1.3 Hz, 1H), 5.89 (d, J=1.3 Hz, 1H), 4.09-4.04 (m, 1H), 3.80 (ddd, J=7.9, 4.2, 1.2 Hz, 1H), 3.53 (s, 1H), 3.44-3.36 (m, 1H), 2.64 (dt, part A of the AB system, J=13.1, 3.9 Hz, 1H), 2.14 (dd, part B of the AB system, J=13.1, 8.0 Hz, 1H). HRMS (Q-TOF): m/z [M]⁺ Calculated C₁₂H₁₀Cl₂O₂: 256.0052, found: 256.0053.

### (1S,2S,4S)-2-chloro-1-(chloromethyl)-7,7-dimethylbicyclo[2.2.1]heptane (2n) 7,8

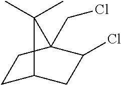

Colorless oil (77 mg, 80%). ¹H NMR (400 MHz, CDCl₃) δ 4.18 (dd, J=8.5, 4.5 Hz, 1H), 3.95 (d, J=10.8 Hz, 1H), 3.52 (d, J=10.8 Hz, 1H), 2.29-2.18 (m, 1H), 2.08 (dd, J=14.0, 8.6 Hz, 1H), 1.80 (m, 3H), 1.53-1.45 (m, 1H), 1.22-1.11 (m, 4H), 0.93 (s, 3H).

### (1S,2R,4R)-2-chloro-1-(chloromethyl)-7,7-dimethylbicyclo[2.2.1]heptane (20)^{7,8}

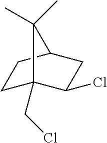

Colorless oil (77 mg, 80%). ¹H NMR (400 MHz, CDCl₃) δ 4.18 (dd, J=8.5, 4.5 Hz, 1H), 3.95 (d, J=10.8 Hz, 1H), 3.52 (d, J=10.8 Hz, 1H), 2.29-2.18 (m, 1H), 2.08 (dd, J=14.0, 8.6 Hz, 1H), 1.80 (m, 3H), 1.53-1.45 (m, 1H), 1.22-1.11 (m, 4H), 0.93 (s, 3H). ¹³C NMR (101 MHz, CDCl₃) 64.3, 54.3, 48.8, 47.6, 46.1, 41.8, 33.0, 26.6, 21.1, 20.4.

### (S)-5-(3-chloroprop-1-en-2-yl)-2-methylcyclohex-2-en-1-one (2p)⁹

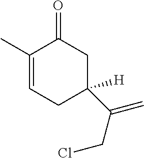

Colorless oil (55 mg, 60%). ¹H NMR (400 MHz, CDCl₃) δ 6.79-6.73 (m, 1H), 5.26 (s, 1H), 5.06 (d, J=1.3 Hz, 1H), 4.09 (s, 2H), 3.07-2.88 (m, 1H), 2.72-2.19 (m, 4H), 1.82-1.77 (m, 3H). ¹³C NMR (101 MHz, CDCl₃) δ 199.1, 146.8, 144.2, 135.8, 115.3, 47.1, 43.2, 38.0, 31.6, 15.8.

### (R)-5-(3-chloroprop-1-en-2-yl)-2-methylcyclohex-2-en-1-one (2q)⁹

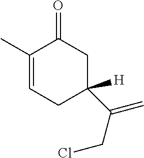

Colorless oil (57 mg, 62%). ¹H NMR (400 MHz, CDCl₃) δ 6.79-6.73 (m, 1H), 5.26 (s, 1H), 5.06 (d, J=1.3 Hz, 1H), 4.09 (s, 2H), 3.07-2.88 (m, 1H), 2.72-2.19 (m, 4H), 1.82-1.77 (m, 3H). ¹³C NMR (101 MHz, CDCl₃) δ 199.1, 146.8, 144.2, 135.8, 115.3, 47.1, 43.2, 38.0, 31.6, 15.8.

### 1,2-Dibromooctane (3a)¹⁰

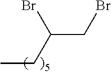

Colorless oil (133 mg, 98%). ¹H NMR (400 MHz, CDCl₃) δ 4.25-4.11 (m, 1H), 3.85 (dd, J=10.3, 4.4 Hz, 1H), 3.63 (t, J=9.9 Hz, 1H), 2.19-2.08 (m, 1H), 1.88-1.68 (m, 1H), 1.64-1.51 (m, 1H), 1.49-1.22 (m, 7H), 0.89 (t, J=6.8 Hz, 3H). ¹³C NMR (101 MHz, CDCl₃) δ 53.3, 36.5, 36.2, 31.7, 28.6, 26.9, 22.7, 14.2.

### trans-1,2-Dibromocyclohexane (3b)¹⁰

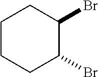

Colorless oil (115 mg, 95%). ¹H NMR (400 MHz, CDCl₃) δ 4.45 (s, 2H), 2.52-2.38 (m, 2H), 1.96-1.73 (m, 4H), 1.60-1.44 (m, 2H). ¹³C NMR (101 MHz, CDCl₃) δ 55.3, 32.1, 22.5.

### trans-2,3-Dibromo-1,2,3,4-tetrahydronaphthalene (3c)¹¹

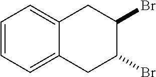 [SMILES: Br[C@@H]1Cc2ccccc2C[C@H]1Br]

White solid (140 mg, 97%). EN: 65-67° C. ¹H NMR (400 MHz, CDCl₃) δ 7.25-7.19 (AA′ part of AA′BB′ system, 2H), 7.14-7.09 (BB′ part of AA′BB′ system, 2H), 4.81-4.72 (m, 2H), 3.99 (dd, A part of AB system, J=17.8, 3.1 Hz, 2H), 3.29 (d, B part of AB system, J=17.8 Hz, 2H). ¹³C NMR (101 MHz, CDCh) δ 130.8, 129.2, 126.9, 49.3, 34.7.

### (1,2-Dibromoethyl)benzene (3d)¹⁰

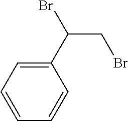

White solid (125 mg, 95%). EN: 73-75° C. ¹H NMR (400 MHz, CDCl₃) δ 7.48-7.31 (m, 5H), 5.15 (dd, J=10.6, 5.5 Hz, 1H), 4.23-3.97 (m, 2H). ¹³C NMR (101 MHz, CDCl₃) δ 138.8, 129.3, 129.0, 127.8, 77.5, 77.2, 76.8, 51.0, 35.2.

### 1-(tert-butyl)-4-(1,2-dibromoethyl)benzene (3e)¹⁰

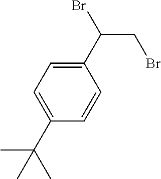

White solid (150 mg, 94%). EN: 60-62° C. ¹H NMR (400 MHz, CDCl₃) δ 7.40 (d, J=8.5 Hz, 2H), 7.34 (d, J=8.5 Hz, 2H), 5.17 (dd, J=10.2, 5.7 Hz, 1H), 4.12-4.00 (m, 2H), 1.33 (s, 9H). ¹³C NMR (101 MHz, CDCl₃) δ 152.4, 135.7, 127.4, 126.0, 51.4, 35.3, 34.9, 31.4.

### 4-(1,2-Dibromoethyl)phenyl acetate (3f)¹²

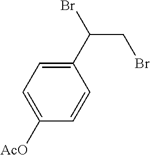 [SMILES: CC(=O)Oc1ccc(C(Br)CBr)cc1]

White solid (148, 92%). EN: 90-92° C. ¹H NMR (400 MHz, CDCl₃) δ 7.42 (d, J=8.6 Hz, 2H), 7.12 (d, J=8.6 Hz, 2H), 5.14 (dd, J=10.7, 5.3 Hz, 1H), 4.07 (dd, J=10.3, 5.3 Hz, 1H), 3.98 (t, J=10.5 Hz, 1H), 2.31 (s, 3H). ¹³C NMR (101 MHz, CDCl₃) δ 169.2, 151.1, 136.2, 129.0, 122.1, 50.2, 35.1, 21.3.

### (1R,2R)-1,2-dibromo-2,3-dihydro-1H-indene (3g)¹³

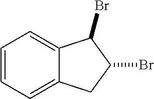 [SMILES: Br[C@@H]1Cc2ccccc2[C@H]1Br]

White solid (132 mg, 96%). EN: 29-31° C. ¹H NMR (400 MHz, CDCl₃) δ 7.49-7.45 (m, 1H), 7.38-7.28 (m, 3H), 5.63 (s, 1H), 4.91-4.80 (m, 1H), 3.82 (dd, J=17.5, 5.2 Hz, 1H), 3.27 (d, J=17.5 Hz, 1H). ¹³C NMR (101 MHz, CDCl₃) δ 140.7, 129.9, 128.1, 125.9, 125.6, 57.9, 54.6, 41.6 (2C signal coincident).

### (E)-1,2-Dibromo-1,2-diphenylethene (3h)¹⁴

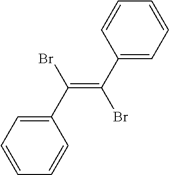 [SMILES: Br/C(=C(/Br)c1ccccc1)c1ccccc1]

White solid (178 mg, 97%). EN: 215-217° C. ¹H NMR (400 MHz, CDCl₃) δ 7.58-7.49 (m, 4H), 7.48-7.32 (m, 6H). ¹³C NMR (101 MHz, CDCl₃) δ 140.9, 129.2, 129.1, 128.5, 118.2.

### trans-10, 11-Dibromo-10, 11-dihydro-5H-dibenzo[a,d][7]annulen-5-one (3i)¹⁵

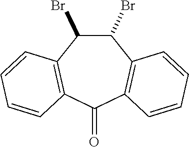 [SMILES: O=C1c2ccccc2[C@@H](Br)[C@H](Br)c2ccccc21]

White solid (180 mg, 98%). EN: 204-206° C. 1H-NMR (400 MHz, CDCl₃): 0=8.09 (dd, J=7.9 Hz, J=1.5 Hz, 2H), 7.57 (dt, J=7.3 Hz, J=1.5 Hz, 2H), 7.50 (dt, J=7.9 Hz, J=1.5 Hz, 2H), 7.41 (dd, J=7.3 Hz, J=1.5 Hz, 2H), 5.80 (s, 2H). ¹³C NMR (100 MHz, CDCl₃) δ 192.3, 138.1, 136.8, 132.9, 131.6, 131.1, 129.7, 52.9.

### 2,3-Dibromo-1,3-diphenylpropan-1-one (3j)¹

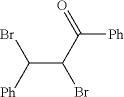 [SMILES: O=C(c1ccccc1)C(Br)C(Br)c1ccccc1]

White solid (171 mg, 93%). EN: 160-162° C. ¹H NMR (400 MHz, CDCl₃) δ 8.17-8.06 (m, 2H), 7.72-7.62 (m, 1H), 7.60-7.51 (m, 4H), 7.47-7.36 (m, 3H), 5.84 (d, J=11.4 Hz, 1H), 5.65 (d, J=11.4 Hz, 1H). ¹³C NMR (101 MHz, CDCl₃) δ 191.3, 138.4, 134.5, 134.4, 129.5, 129.2, 129.1, 129.0, 128.5, 49.9, 47.0.

### (1R(S),2S(R),4R(S),9R(S))-2,9-dibromo-1,2,3,4-tetrahydro-1,4-methanonaphthalene (3k)¹⁶

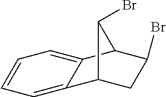 [SMILES: BrC1CC2c3ccccc3C1C2Br]

White solid (149 mg, 99%). EN: 76-78° C. ¹H NMR (400 MHz, CDCl₃) δ 7.25-7.12 (m, 4H), 4.18-4.13 (m, 1H), 3.80 (dd, J=8.0, 4.6, 1.3 Hz, 1H), 3.75 (bs, 1H), 3.54-3.49 (m, 1H), 2.87 (dt, part A of system AB, J=13.4, 4.2 Hz, 1H), 2.21 (dd part B of system AB, J=13.4, 8.0 Hz, 1H). ¹³C NMR (101 MHz, CDCl₃) δ 143.7, 143.1, 128.0, 127.4, 121.9, 121.5, 56.6, 55.7, 51.2, 45.2, 36.7.

### (1R(S),2S(R),4R(S),9R(S))-2,9-dibromo-5,8-dimethoxy-1,2,3,4-tetrahydro-1,4-methanonaphthalene (3I)

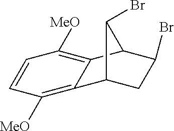 [SMILES: COc1ccc(OC)c2c1C1CC(Br)C2C1Br]

Pale yellow oil (175 mg, 97%). ¹H NMR (400 MHz, CDCl₃) δ 6.65 (d, J=9.0 Hz, 1H), 6.61 (d, J=9.0 Hz, 1H), 4.13-4.09 (m, 1H), 3.97-3.94 (m, 1H), 3.83-3.77 (m, 4H), 3.76 (s, 3H), 3.73-3.67 (m, 1H), 2.83 (dt, part A of system AB, J=13.4, 4.1 Hz, 1H), 2.20 (dd, part B of system AB, J=13.4, 8.0 Hz, 1H). ¹³C NMR (101 MHz, CDCl₃) δ 147.8, 147.3, 132.7, 131.9, 111.1, 110.3, 56.1, 55.9, 55.7, 52.8, 47.7, 45.1, 36.2. HRMS (Q-TOF): m/z [M]⁺ calculated C₁₃H₁₄Br₂O₂: 359.9355, found: 359.9355.

### (5R(S),6S(R),8R(S),10R(S))-6,10-dibromo-5,6,7,8-tetrahydro-5,8-methanonaphtho[2,3-d][1,3]dioxole (3m)

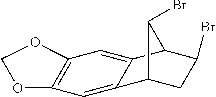 [SMILES: BrC1CC2c3cc4c(cc3C1C2Br)OCO4]

White solid (152 mg, 88%). EN: 94-96° C. ¹H NMR (400 MHz, CDCl₃) δ 6.73 (s, 1H), 6.69 (s, 1H), 5.93 (d, J=1.3 Hz, 1H), 5.90 (d, J=1.3 Hz, 1H), 4.14-4.10 (m, 1H), 3.74 (dd, J=8.0, 4.6, 1.3 Hz, 1H), 3.65 (bs, 1H), 3.44-3.37 (m, 1H), 2.82 (dt, part A of system AB, J=13.2, 4.2 Hz, 1H), 2.16 (dd, part B of system AB, J=13.2, 8.0 Hz, 1H). ¹³C NMR (101 MHz, CDCl₃) δ 147.1, 146.6, 137.2, 136.2, 103.9, 103.3, 101.1, 56.5, 55.1, 51.1, 45.3, 36.8. HRMS (Q-TOF): m/z [M]⁺ calculated C₁₂H₁₀Br₂O₂: 343.9042, found: 343.9043.

### (1S,2S,4S)-2-bromo-1-(bromomethyl)-7,7-dimethylbicyclo[2.2.1]heptane (3n)¹⁷

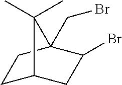

Brown solid (127 mg, 90%). EN: 63-65° C. ¹H NMR (400 MHz, CDCl₃) δ 4.26 (dd, J=8.6, 4.6 Hz, 1H), 3.77 (d, J=9.9 Hz, 1H), 3.48 (d, J=9.9 Hz, 1H), 2.43 (ddd, J=14.3, 7.9, 4.6 Hz, 1H), 2.16 (dd, J=14.3, 8.6 Hz, 1H), 1.99 (t, J=4.4 Hz, 1H), 1.93 (dd, J=13.1, 4.4 Hz, 1H), 1.79 (dtd, J=11.9, 7.5, 4.1 Hz, 1H), 1.61-1.51 (m, 2H), 1.21 (s, 3H), 0.94 (s, 3H). ¹³C NMR (101 MHz, CDCl₃) δ 56.8, 53.2, 49.5, 48.4, 42.2, 37.4, 34.6, 26.5, 21.1, 20.5.

### (1S,2R,4R)-2-bromo-1-(bromomethyl)-7,7-dimethylbicyclo[2.2.1]heptane (30)¹⁷

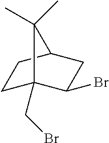

Brown solid (127 mg, 90%). EN: 63-65° C. ¹H NMR (400 MHz, CDCl₃) δ 4.26 (dd, J=8.6, 4.6 Hz, 1H), 3.77 (d, J=9.9 Hz, 1H), 3.48 (d, J=9.9 Hz, 1H), 2.43 (ddd, J=14.3, 7.9, 4.6 Hz, 1H), 2.16 (dd, J=14.3, 8.6 Hz, 1H), 1.99 (t, J=4.4 Hz, 1H), 1.93 (dd, J=13.1, 4.4 Hz, 1H), 1.79 (dtd, J=11.9, 7.5, 4.1 Hz, 1H), 1.61-1.51 (m, 2H), 1.21 (s, 3H), 0.94 (s, 3H). ¹³C NMR (101 MHz, CDCl₃) ö 56.8, 53.2, 49.5, 48.4, 42.2, 37.4, 34.6, 26.5, 21.1, 20.5.

### (S)-5-((R)-1,2-dibromopropan-2-yl)-2-methylcyclohex-2-en-1-one (3p) and(S)-5-((S)-1, 2-dibromopropan-2-yl)-2-methylcyclohex-2-en-1-one mixture (3q)¹

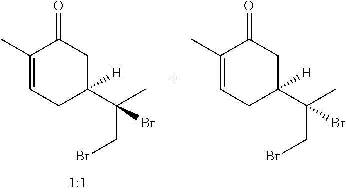

Colorless oil (142 mg, 92%). ¹H NMR (400 MHz, CDCl₃) δ 6.79-6.70 (m, 2H), 3.99-3.91 (m, 2H), 3.85 (d, J=10.4 Hz, 1H), 3.81 (d, J=10.5 Hz, 1H), 2.74-2.25 (m, 10H), 1.88 (s, 3H), 1.86 (s, 3H), 1.80 (s, 3H), 1.79 (s, 3H). ¹³C NMR (101 MHz, CDCl₃) δ 198.6, 198.2, 143.8, 143.5, 135.5, 135.4, 71.2, 71.1, 42.4, 42.1, 40.8, 40.73, 40.68, 40.1, 28.9 (2C), 28.5, 28.0, 15.72, 15.71.

### Mixture of (R)-5-((R)-1,2-dibromopropan-2-yl)-2-methylcyclohex-2-en-1-one (3r) and (R)-5-((S)-1,2-dibromopropan-2-yl)-2-methylcyclohex-2-en-1-one (3s)¹

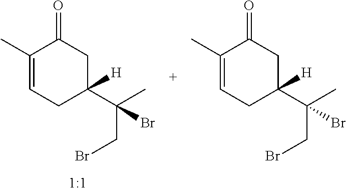

Colorless oil (142 mg, 92%). ¹H NMR (400 MHz, CDCl₃) ö 6.81-6.71 (m, 2H), 4.00-3.92 (m, 2H), 3.86 (d, J=10.4 Hz, 1H), 3.82 (d, J=10.5 Hz, 1H), 2.72-2.30 (m, 10H), 1.90 (s, 3H), 1.88 (s, 3H), 1.80 (s, 3H), 1.79 (s, 3H). ¹³C NMR (101 MHz, CDCl₃) ö 198.6, 198.2, 143.8, 143.6, 135.5, 135.4, 71.2, 71.1, 42.4, 42.1, 40.8, 40.72, 40.67, 40.1, 28.9 (2C), 28.4, 28.0, 15.73, 15.71.

### Dibromo-kolesterol (3t)¹⁸

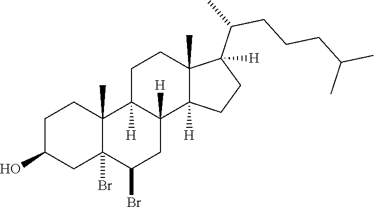

Brown viscous (88%). ¹H NMR (400 MHz, CDCl₃) ö 4.85 (dd, J=12.5, 5.0 Hz, 1H), 4.24-4.15 (m, 1H), 3.16 (bs, 1H), 2.82 (d, J=16.6 Hz, 1H), 2.49 (dd, J=16.6, 4.5 Hz, 1H), 2.29-2.19 (m, 1H), 2.12-0.58 (m, 40H). ¹³C NMR (101 MHz, CDCl₃) ö 89.8, 69.2, 56.2, 56.1, 55.2, 47.4, 45.8, 42.7, 41.9, 39.6, 39.5, 37.2, 36.7, 36.1, 35.8, 30.8, 30.2, 28.2, 28.0, 24.1, 23.8, 22.8, 22.6, 21.3, 20.4, 18.7, 12.2.

With the effective oxidative chlorination and bromination method of olefins in the presence of Selectfluor and TBAX (Cl, Br), which have all these features, all the disadvantages of the molecular halogenation reagents (Cl₂, Br₂) and metal halides (MXn) are eliminated. The need for halogen reagents and the use of metal halides, which are not preferred in terms of medicinal chemistry, will also be eliminated.

The method of the invention will enable the synthesis of halogenated products with high efficiency and selectivity, derivatization of natural chiral compounds and the use of safe solvents in mild conditions.

With the inventive system, improvements are also achieved in terms of environmental impact, energy use, cost and labor force in the field of the invention.

1. Efficient oxidative chlorination and bromination of olefins in the presence of selectfluor and TBAX (Cl, Br), characterized in that, olefins are chlorinated using TBACl+Selectfluor.

2. Efficient oxidative chlorination and bromination of olefins in the presence of selectfluor and TBAX (Cl, Br), characterized in that, olefins are brominated using TBAB+Selectfluor.
